(19) United States
(12) Patent Application Publication
(10) Pub. No.: US 2008/0199516 A1
(43) Pub. Date: Aug. 21, 2008

(54) Encapsulation Of Lipid-Based Formulations In Enteric Polymers

(75) Inventor: Stephen Mark Mcallister, Harlow Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/996,352

(22) PCT Filed: Jul. 24, 2006
(86) PCT No.: PCT/EP2006/007387

§ 371 Date: Jan. 22, 2008

Related U.S. Application Data
(60) Provisional application No. 60/702,669, filed on Jul. 26, 2005.

(51) Int. Cl. A61K 9/50

(52) U.S. Cl. 424/455

(57) ABSTRACT

A microcapsule comprising a lipid-based core that is encapsulated in an enteric polymer shell providing enhanced bioavailability of a sparingly water-soluble drug as well as modulated release of the drug, wherein the microcapsule is, in one embodiment, prepared by a centrifugal coextrusion process. The lipid-based core comprises lipidic carriers, either liquid or solid (melting point<1000C), that would provide adequate drug solubilization and is compatible with the enteric shell materials.

### FIELD OF THE INVENTION

The present invention relates generally to microcapsules containing a lipid-based formulation and methods for making such microcapsules. More particularly, the present invention relates to microcapsules having a lipid-based formulation encapsulated in an enteric polymer shell and methods for making such microcapsules.

### BACKGROUND OF THE INVENTION

Oral administration is the preferred route for administration of therapeutic agents, especially medications taken on a daily outpatient basis. Often the oral absorption characteristics of many of the compounds are poor and they have to be formulated using delivery technologies to enhance dissolution, alter the time course of absorption, or target absorption in a particular region of the gastrointestinal tract.

Oral drug delivery systems can be classified into two categories: modified release delivery systems and bioavailability-enhanced delivery systems. Bioavailability-enhanced delivery systems have attracted a lot of interest lately because high throughput screening processes often identify insoluble drug candidates with poor bioavailability. The majority of hydrophobic drugs are not easily absorbed in the gastrointestinal tract due to limitations of solubility and dissolution in the gastrointestinal fluids. These bioavailability enhanced delivery systems often consist of molecular dispersions of the sparingly water-soluble drugs in lipid-based carriers, preferably carriers that can spontaneously emulsify (self-emulsifying formulations). These carriers can deliver a drug in a presolubilized form for rapid absorption. The self-emulsifying lipid-based formulations described herein are usually encapsulated in soft and hard gelatin capsules. Several formulations are currently on the market, e.g., Sandimmun®/Neoral® (cyclosporin microemulsion), Norvir® (Ritnovir) and Fortovase® (Saquinavir).

Modulating or controlling the rate of drug release of these bioenhanced formulations may provide many important benefits both therapeutically and commercially. The USP definition of a modified release dosage form is one in which the drug release characteristics of time, course and/or location are chosen to accomplish therapeutic or convenience objectives not offered by conventional dosage forms.

Encapsulation of various compounds and formulations is known in the art. By way of example, a brief description of the centrifugal extrusion process for encapsulation of lipophilic cores in a variety of shell materials is provided in U.S. Pat. Nos. 3,310,612; 3,389,194; 4,888,140; and, 5,348,803. It is important to note, however, that these shell materials previously known for use in encapsulation by centrifugal extrusion lack the ability to modulate release of a therapeutically active agent. Encapsulation of aqueous cores in a polymeric shell is described in U.S. Pat. No. 5,330,835. Details on encapsulation of insoluble microparticulates composed of biodegradable polymers in enteric polymers is described in U.S. Pat. Nos. 5,382,435 and 5,505,976. Another U.S. Pat. No. 5,246,636 describes a method of forming multi-walled capsules. Alternatively, microspheres prepared by vibratory excitation are known and described in U.S. Pat. Nos. 6,197,073; 5,420,086; and 5,472,648 and European Patent No. 1 467 221. Encapsulation of such microspheres, thereby creating microcapsules, is described in a publication entitled, Preparation of Monodisperse Controlled Release Microcapsules, Brandau, International Journal of Pharmaceutics 242 (2002) pp. 179-184.

Successful encapsulation of self-emulsifying formulations in soft and hard gelatin capsules is difficult and depends on many factors, including: identifying appropriate shell materials, preventing unwanted water exchange (between shell and core), achieving acceptable brittleness and softness specifications. Even when successful, such encapsulation has, historically, resulted in a product with disadvantages such as poor product handling qualities, lengthy processing time, and most importantly, the inability to modulate the release profile.

The centrifugal extrusion encapsulation process is currently used for manufacture of capsules containing fragrance, vitamins, etc., using gelatin, alginates or fats as the shell materials. Such applications are not typically focused on modulating the release profile of the active ingredients therein.

It is therefore desirable from a processing, performance, stability and cost perspective to evaluate alternate methods for encapsulating lipid-based core formulations (with varying HLB values) of sparingly water soluble therapeutic agents with shell materials that can overcome these limitations.

### SUMMARY OF THE INVENTION

The present invention provides microcapsules having a lipid-based core formulation that is encapsulated within a polymer shell. The lipidic cores comprise lipidic carriers and at least one sparingly water soluble therapeutic agent. The lipidic carriers are in the form of liquids or solids (melting point<100° C.) that would provide adequate drug solubilization and are compatible with the shell material.

Suitable shell materials for use in the present invention include those materials that are able to modulate release characteristics of a therapeutic active, such as functional polymers. The functional polymers suitable for use in the present invention include enteric, film-forming polymers. Such enteric polymers are good film formers that can resist dissolution in an acidic environment (pH from about 1 to about 3) like those encountered in the stomach but can dissolve rapidly in the more alkaline environment (pH>5) of the small intestine. The enteric protection is required to prevent gastric mucosal irritation or protect a drug that is unstable in an acidic environment or to delay or modulate release for local delivery in the intestine.

In one embodiment, the lipid-based formulations described herein are encapsulated in an enteric polymer shell using the centrifugal extrusion process to produce microcapsules (<2 mm). The process is fairly simple and robust in terms of producing particles in a desired size range with a high drug loading and, provides operating versatility from a standpoint of handling different types of core and shell materials. Since the process is continuous, there are minimal start-up and shutdown steps, leading to higher production output when compared with standard batch operations. Another advantage of the coextrusion process pertains to the capsule morphology. Centrifugal extrusion gives true core/shell morphology where the capsule consists of a single droplet of core material surrounded by a shell.

In another embodiment, the lipid-based formulations described herein are encapsulated in an enteric polymer shell using a double nozzle, vibratory excitation process to produce microcapsules. Such microcapsules consist of a solid shell surrounding a liquid or solidified core.

### BRIEF DESCRIPTION OF THE DRAWINGS

FIG. 1 is a side view of a centrifugal encapsulation apparatus for making microcapsules according to the present invention;

FIG. 2 is an optical micrograph of a microcapsule with a lipid-based formulation encapsulated in an enteric polymer shell according to the present invention;

FIG. 3 is another optical micrograph of a microcapsule with a lipid-based formulation encapsulated in an enteric polymer shell according to the present invention;

FIG. 4 is a SEM micrograph of a microcapsule with a lipid-based formulation encapsulated in an enteric polymer shell according to the present invention;

FIG. 5 is a graph showing the release characteristics of the microcapsules of the present invention, which comprise a lipid-based formulation encapsulated in an enteric polymer shell, when placed in a dissolution medium at an acidic pH (simulated gastric fluid) and an alkaline pH (simulated intestinal fluid) and represented as a function of concentration versus time; and

FIG. 6 is an optical micrograph of a microcapsule according to the present invention that is exposed to simulated gastric fluid showing no rupture of shell material.

FIG. 7 is a photograph taken using an optical microscope with background lighting through a polarizing filter of a microcapsule according to the present invention prepared by double nozzle vibratory excitation.

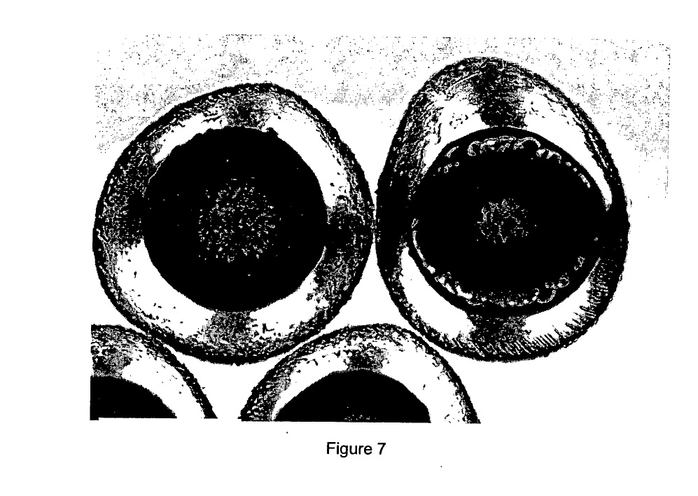

### DETAILED DESCRIPTION OF THE INVENTION

The present invention relates to microcapsules comprising lipid-based formulations encapsulated within an enteric polymer shell. Oral formulations comprising the mirocapsules of the present invention offers twin advantages of bioavailability enhancement and modified release. The present invention also provides a process for mass production of microcapsules. The microcapsules of the present invention have a distinct core/shell morphology. The microcapsules exhibit negligible dissolution in an acidic (pH<3) environment, yet also exhibited rapid drug release and dissolution in a more alkaline (pH>5) environment.

### Lipidic Core Formulation

The microcapsules comprise a lipid-based core material that is encapsulated within a polymer shell. The lipidic core comprises lipidic carriers forming a dispersion matrix, and at least one sparingly water-soluble therapeutic agent. That is, the lipidic core of the present invention is either a liquid or solid molecular dispersion of a sparingly water-soluble drug. The melting point of the lipidic carriers used in the dispersion matrix is <100° C. The lipidic carriers provide adequate drug solubilization at temperatures much below the melting temperature of the drug, and are compatible with the shell material. The lipidic carriers also provide adequate drug solubilization in the intestinal milieu, without precipitation and/or agglomeration, and with concomitant improvement in bioavailability. In addition, some of the lipidic carriers in the dispersion matrix can enhance drug bioavailability by increasing intestinal permeability (e.g., P-gp inhibition).

The lipidic carriers include medium or long chain fatty acid esters and a lipid-based surfactant. Suitable lipid-based surfactants and fatty acid esters are those in which the sparingly water-soluble component or drug has adequate solubility at a temperature below the melting point of the drug. Other components that may be added to the lipid matrix include, for example, an adjuvant to enhance drug solubility.

### Lipid-Based Esters

The lipid-based esters are medium or long chain fatty acid esters, such as mixed glycerides that have adequate drug solubility and the ability to modulate rigidity of the lipidic dispersion matrix. These mixed glycerides are derived from edible oils and fats obtained from suitable fatty acid sources. Suitable fatty acid sources include any vegetable or animal sources, such as, but not limited to, cottonseed oil, palm oil, lard, tallow or any combinations thereof. The concentration of fatty acid ester or mixed glycerides in the lipid-based core is about 75% to about 99.99% by total core weight of the lipidic core. In one embodiment, the concentration of fatty acid ester or mixed glycerides in the lipidic core is about 80% to about 95% by total weight of the lipidic core.

The fatty acid esters are mixed glycerides, that include medium and long chain fatty acids that are either solids or liquids at room temperature. The medium chain triglycerides that may be used in the present invention include, for example, caprylic/capric triglyceride (Crodamol® GTC/C), glyceryl tricaprylate/caprate (Miglyol® 810 and 812), Neobee® M5, corn oil, peanut oil, glycerol mono-oleate (Pecol® FCC), Labrafac® CC, or any combinations thereof. The long chain triglycerides that may be used in the present invention include, for example, glycerol monostearate (Myverol® 18-07, 18-85, Imwitor® 491), glycerol palmitostearate, or any combinations thereof. Other mixed glycerides that may be used include, but are not limited to, fully hydrogenated vegetable oils obtained from a variety of sources (Sterotex® K, NF and HM), partially hydrogenated vegetable oils (Dynasan® P60, Softisan® 154, Paramount® C, Duramel®, etc.), or any combinations thereof.

The mixed glycerides used can serve as solubilizing agent, emulsifying agent and suspending agent for the dispersed or dissolved drug. The high molecular weight mixed glycerides can also act as a stiffening agent in the core and inhibit the compound's molecular mobility in the dispersion matrix, thus improving the physical and chemical stability of the compound during storage. Most of the mixed glycerides used herein are described in detail in the Handbook of Pharmaceutical Excipients, published jointly by the American Pharmaceutical Association and The Pharmaceutical Society of Great Britain, incorporated herein by reference.

Suitable medium chain mixed glycerides used in the lipidic core of the present invention include, but are not limited to, Miglyol® 812 or 810, commercially available from Condea Chemicals, Germany, Pecol® FCC and Labrafac® CC commercially available from Gattefosse Corporation, West Kindermack Road, New Jersey, capric triglycerides (Crodamol® GTC/C), Neobee® M5, corn oil and peanut oil, which can be obtained from Croda, Parsippany, N.J., or any combinations thereof.

Suitable high molecular weight mixed glycerides include, but are not limited to, glycerol monostearate (GMS), glycerol palmitostearate, hydrogenated vegetable oils, or any combinations thereof. Examples of GMS that can be used in the lipidic core of this invention include Myverol® 18-07 or Imwitor® 491. Myverol® 18-07 is food grade glycerol monostearate commercially available from Quest International, Hoffman Estates, Illinois. Imwitor® 491 is pharmaceutical grade glycerol monostearate that is commercially available from Sassol, Germany. Both of these products are available as small microbeads that are free flowing, have an average molecular weight of about 350, and a melting point in the range of 50° C. to 70° C.

Suitable glycerol palmitostearate useful as a stiffening agent in the solid dispersions of the present invention include, but are not limited to, Precirol® ATO5 commercially available from Gattefosse Corporation, West Kindermack Road, New Jersey. Precirol® ATO5 is available as a fine white powder with faint odor and a melting point in the range of 52° C. to 55° C.

Suitable hydrogenated vegetable oil (mixed glycerides) useful as a stiffening agent in the solid dispersions of this invention include, but are not limited to, Sterotex®HM, Sterotex®K, Sterotex®NF, or combinations thereof, which are commercially available from Abitec Corporation, Janesville, Wis. Hydrogenated vegetable oils are available as fine powder, flakes or pellets. The color of the material depends on the manufacturing process. In general, the material is white to yellowish-white and the melting point is in the range of 60° C. to 70° C.

Suitable partially hydrogenated vegetable oil (mixed glycerides) used in the lipidic matrix of this invention include, but are not limited to, Paramount®C, Duramel®, Dynasan® P60, Softisan® 154, or any combinations thereof, which are available as a semi-solid waxy material from Abitec Corporation, Janesville, Wis.

### Lipid-Based Surfactants

The lipid-based surfactants used herein are identified by their HLB value, where the HLB value is a measure of their hydrophobic or hydrophilic nature. The concentration of surfactant is about 0.1% to about 25% by total core weight of the lipidic core. In one embodiment, the concentration of surfactant present in the lipidic core is about 5% to about 25% by total weight of the lipidic core. The lipidic surfactant in the core has two important functions. It acts as a solubilizer for the lipophilic drug and as an emulsifier for precipitated drug particles in an aqueous environment. Suitable surfactants for use in the lipidic core of the present invention include, but are not limited to, polyglycolized glycerides (Gelucire®), vitamin E tocopherol polyethylene glycol succinate (vitamin E TPGS®), polyoxyethylene castor oil derivatives (Cremophor®), polyoxyethylene alkyl ethers (Myrj®), sorbitan fatty acid esters (Span®), polyoxyethylene sorbitan fatty acid esters (Tween®) or any combinations thereof. Particularly preferred surfactants include one or more of glycerides (Gelucire®), vitamin E TPGS, or combinations thereof. Additional lipid-based surfactants that can be used in the core of the present invention are described in detail in the Handbook of Pharmaceutical Excipients.

Suitable polyglycolized glycerides useful as a lipid-based surfactant in the lipidic matrix of the present invention include, but are not limited to, lauroyl macrogoglyceride and stearoyl macrogoglyceride (Gelucire® 44/14 and Gelucire® 50/13 respectively, sold by Gattefosse Corporation, West Kindermack Road, New Jersey), or combinations thereof. These surfactants disperse in an aqueous media forming micelles, microscopic vesicles or globules. Lauroyl macrogoglycerides and stearoyl macrogoglycerides are digestible GRAS materials that are available as a semi-solid waxy material, granules or pastilles with HLB values of about 14 and about 13 and melting points of about 44° C. and about 50° C., respectively.

Vitamin E TPGS (Sold by Eastman, Kingsport, Tenn.) is water soluble derivative of vitamin E prepared by esterification of the acid group of d-α-tocopheryl succinate by polyethylene glycol 1000. Structurally, it has a dual nature of lipophilicity and hydrophilicity, similar to a surface-active agent and can act as a solubilizer, emulsifier and absorption enhancer (P-gp inhibition). Vitamin E TPGS has a high HLB value in the range of about 15 to about 19.

Examples of suitable polyoxyethylene castor oil derivatives suitable for use as a lipid-based surfactant in the lipidic matrix of this invention include polyoxyl 35 castor oil, polyoxyl 40 or 60 hydrogenated castor oil (sold by BASF Corporation, Mount Olive, N.J. under the tradename Cremophor® EL, Cremophor® RH 40 or 60, respectively), or any combinations thereof. These polyoxyethylene castor oil derivatives are either liquids or solids that have an HLB value in the range of about 10 to about 17.

Polyoxyethylene stearates, useful as lipid-based surfactants in the present invention, are non-ionic surfactants, which include, for example, polyethoxylated derivatives of stearic acid and particularly those sold under the trade name Myrj®, by Uniqema, New Castle, Del. These surfactants are typically available as waxy solids or pastes, have HLB values in the range of about 10 to about 15, and a melting point in the range of 28° C. to 57° C.

### Optional Solubilization Enhancer

The lipid-based core of the present invention may also have a solubilization enhancer. Generally, the concentration of the solubilization enhancer is about 0.01% to about 10% by total core weight of the lipidic core.

Exemplary solubilization enhancers that are suitable for the lipid-based core of the present invention include, but are not limited to, mid-weight polyethylene glycol (PEG) having molecular weight from 1000 to 8000. In one embodiment, the solubilization enhancer is polyethylene glycol with an average molecular weight from 2000 to 6000. Suitable PEG's for use in the lipidic core of the present invention include, but are not limited to, PEG 3350 and PEG 6000 available from Union Carbide Corporation, Danbury, Conn.

### Sparingly Water-Soluble Drug

A drug, specifically a sparingly water-soluble drug, is present in the lipid-based core of the present invention from about 0.01% to about 20% of the total core weight. In one embodiment, the concentration of sparingly water-soluble drug present in the lipidic core is about 1% to about 10% of the total weight of the lipidic core. In yet another embodiment, the sparingly water-soluble drug is present in the lipidic core in an amount of about 1% to about 5% of the total weight of the lipidic core. Examples of sparingly water-soluble compounds are those that have a solubility in water of less than 100 g/mL at 25° C. Such compounds have poor oral bioavailability and include lipophilic drugs, vitamins, and hormones. These compounds include steroids, steroid antagonists, non-steroidal anti-inflammatory agents, antifungal agents, antibacterial agents, antiviral agents, anticancer agents, anti-hypertensive agents, anti-oxidant agents, anti-epileptic agents, anti-depressant agents, and non-peptide enzyme inhibitors among others.

The microcapsule payload (core content) is about 10% to about 80% by total weight of the capsule (“capsule weight”). In one embodiment the microcapsule payload is about 20% to about 60% of the capsule weight. The loading is controlled by setting the feed rate of the liquid core and the shell material during processing that provides the desired dry (after removal of solvent) payload.

### Enteric Polymer Shell Formulation

An important aspect of the present invention is the enteric polymer used to form the shell of the microcapsules. The enteric polymers suitable for use in the present invention are good film formers that can resist dissolution in an acidic environment (i.e., a pH of about 1 to about 3) like those encountered in the stomach, but dissolve rapidly in the more alkaline environment (pH>about 5) of the small intestine.

Examples of enteric polymers useful in the present invention include, but are not limited to, cellulose derivatives such as cellulose acetate phthalate (CAP), hydropropyl methylcellulose phthalate (HPMCP-50 or HPMCP-55), hydroxypropyl methylcellulose acetate succinate (HPMCAS), alkali-soluble acrylic copolymers (Eudragit® L series and Eudragit® S series), polyvinyl acetate phthalate (PVAP), alginates, or any combinations thereof. Depending upon the desired release profile, it may be required to combine these enteric polymers with insoluble (under pH conditions encountered in the gastrointestinal tract) film-forming polymers to modulate release from the microcapsules. These insoluble polymers can either be swellable (at pH>about 5) or permeable (regardless of pH). Permeable acrylic copolymers include, for example, Eudragit® RS and RL. Swellable acrylic copolymers include, for example, Eudragit NE. Examples of permeable cellulose-based polymers include, for example, cellulose acetate (CA) and ethyl cellulose (EC). Swellable cellulose-based polymers include, for example, hydroxypropyl cellulose (Klucel®) and methylcellulose (Methocel®). Enteric and non-enteric polymers are described, more particularly, in the Handbook of Pharmaceutical Excipients.

The pH-solubility characteristics of the cellulose-based enteric polymers used herein can be controlled by varying the phthalate content. Various grades of HPMCP are available with varying degree of substitution, for example HPMCP-50 dissolves at pH 5 and above, whereas HPMCP-55 dissolves at pH above 5.5, and cellulose acetate phathalate (CAP) dissolves at pH>6. These enteric polymers are available, for example, from Shinetsu, Tokyo, Japan.

The permeability of cellulose esters (e.g., cellulose acetate) used depends upon the degree of substitution and carbon chain length of the substituting groups. Increasing the degree of substitution with acetyl group decreases film permeability. Cellulose acetate (CA) is sold by Eastman, Kingsport, Tenn. and FMC Corporation, Princeton, N.J. The permeability of ethyl cellulose (EC) is controlled by the degree of substitution of the cellulose group with ethoxyl groups. Increasing the degree of substitution with ethoxyl group increases the permeability characteristics of the polymer film. EC is sold under the trade name Aquacoat® (FMC Corporation, Princeton, N.J.) and Surelease® (Colorcon, West Point, Pa.).

The different acrylic copolymers (Eudragit® series) offer a range of physicochemical properties depending upon the ester substitution in the chemical structure that determines their pH-solubility and water permeability characteristics. The Eudragit® polymers are made by Rohm Pharma (Dramstadt, Germany). Polyvinyl Acetate Phthalate (Sureteric®) is a specially blended combination that can be used as a substitute for acrylic-based polymers.

### Optional Components

Additional polymers may be incorporated in the enteric shell formulation as gelling agents in the polymer solution to accelerate capsule formation during solvent removal (or drying) process include water-soluble resins such as alginates, carrageenan, gelatin, poly(ethylene oxide), polyvinyl alcohol (PVA), cellulose derivatives such as carboxymethyl cellulose sodium (CMCS), hydroxyethyl cellulose (Natrasol®), hydroxypropylmethyl cellulose (HPMC), hydroxylpropyl cellulose (HPC) or any combinations thereof. The preferred gelling agent includes carragenan, gelatin, alginates, and polyethylene oxide (PEO). The one or more polymers used as gelling agents herein form a gel network based on thixotrophy.

Plasticizers that may be added to the shell solution to modulate flexibility of the polymer film include, but are not limited to, glycerol, polyethylene glycol, triacetin, diethyl phthalate, dibutyl sebecate, esters of citric acid, or any combinations thereof.

In addition, pigments, such as titanium dioxide and FD&C lakes and dyes, can be incorporated in the shell solution to impart color to the microcapsules.

### Method of Making

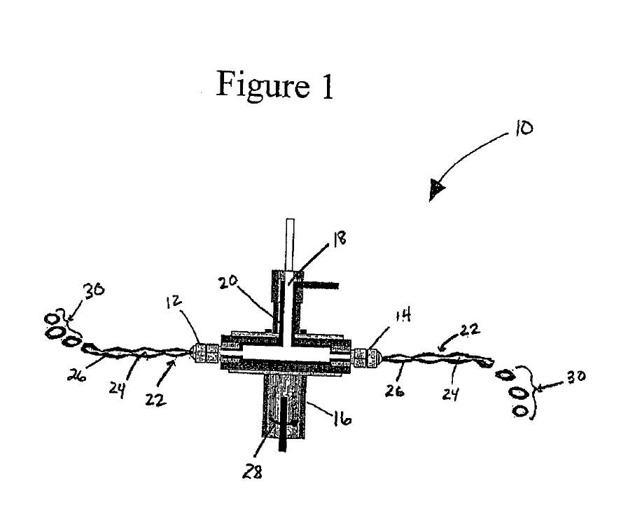

In one embodiment of the present invention, the microcapsules are prepared by a centrifugal coextrusion process. A centrifugal extrusion apparatus is represented generally in FIG. 1, by reference numeral 10. The centrifugal extrusion process is a liquid coextrusion process utilizing concentric nozzles 12, 14 located on the outer circumference of a rotating cylinder 16. A liquid core material is pumped through the inner orifice 18 and through the outer orifice 20 to form coextruded rods 22 of the core material 24 surrounded by shell material 26. As the device rotates, as shown by arrow 28, the extruded rods break into droplets by centrifugal force to form capsules 30.

The centrifugal coextrusion process produces microcapsules in a desired size range with a high payload and provides operating versatility from a standpoint of handling different types of core and shell compositions. Since the process is continuous, there are minimal start-up and shutdown steps, leading to higher production output when compared with standard batch operations. In addition, the centrifugal coextrusion process gives true core/shell morphology where the microcapsule consists of a single droplet of core material surrounded by a distinct shell. This morphology exhibits advantages in terms of improved stability and release profile when compared to a microsphere or micromatrix morphology. The method is capable of handling both polar and non-polar materials in the form of liquid, melts or dispersed solids. A variety of shell compositions can be used depending on end use, to provide a way to control the release characteristics of the capsules.

In one embodiment the microcapsules of the present invention may be prepared by the following method. Initially, the lipidic carrier(s) is heated to a temperature that is about 10° C. to about 20° C. above its melting point (for solids) or to a sufficiently high temperature for liquids (preferably from 60° C. to 80° C.) and dissolving the drug into the carrier(s) by continuous stirring under a nitrogen blanket. The concentration of the active material in the carrier(s) may range from about 0.01% to about 20%, in one embodiment from about 5% to about 10%, based on the total weight of the lipidic core. The viscosity of the lipidic core having the dissolved or dispersed drug is sufficiently low to form droplets when the core material is extruded from the nozzles. The viscosity of the drug/carrier blend may range from about 1 to about 20 poise, and in another embodiment may range from about 5 to about 10 poise.

The enteric polymer shell formulation is next dissolved in a solvent system comprising water, sodium hydroxide, glycerin and trace amounts of Tween® (polysorbate 80). The concentration of sodium hydroxide in the solvent system may range from about 1% to about 10% w/w, in one embodiment from about 2% to about 5% w/w. The concentration of glycerin in the solvent medium may range from about 1% to about 5% w/w, in one embodiment from about 1% to about 2% w/w. The pH of the solution is adjusted to about 5.6 with about 10% glacial acetic acid. The solids content (polymer concentration) of the shell solution is varied for the different polymers used therein and primarily dependent on their molecular weight. Appropriate solids content is determined by resultant viscosity and “stringiness” of the solution. That is, the solids content is adjusted such that the extruded stream can break into droplets without excessive tailing or stringing between the individual capsules. The solids concentration (total combined enteric polymer and gelling agent concentration) may range from about 10% to about 30%, in one embodiment from about 15% to about 25%, by weight of the shell solution. The concentration of gelling agent in the enteric shell formulation may range from about 0.5% to about 5% of the solids concentration, in one embodiment from about 1% to about 2% of the solids concentration. The amount plasticizer in the enteric shell formulation may range from about 1% to about 5%, and in one embodiment from about 2% to about 3%, by weight of the shell solution. The concentration of dyes and pigment in the enteric shell formulation may range from about 1% to about 2% by weight of the shell solution.

Referring again to FIG. 1, to form microcapsules the core material is then pumped through the inner orifice18 and the shell solution is pumped through the outer orifice 20. The feed rate of the core material may range from about 10 to about 60 g/min, in one embodiment from about 40 to about 50 g/min. The feed rate of shell solution may range from about 10 to about 40 g/min in one embodiment from about 20 to about 30 g/min. The core material and the shell solution are pumped using a positive displacement pump (not shown) to accurately control the feed rates. Nozzles 12 and 14 can range in size from an inner diameter of about 0.010 inch (corresponding to an outer diameter of about 0.015 inch) to an inner diameter of about 0.060 inch (corresponding to an outer diameter of about 0.080 inch). One of skill in the art would understand that the choice of nozzle size is dependent on the target microcapsule size.

In addition, the speed of the rotating cylindrical head 16 is varied to control the microcapsule size, with higher speed resulting in smaller microcapsules 30. The speed of the rotating cylindrical head 16 may range from about 200 rpm to about 2000 rpm with higher speeds resulting in the formation of smaller microcapsules. The in one embodiment the rotational speed is from about 500 rpm to about 1500 rpm. Feed rates are used to adjust the capsule payload and to set production rates.

The capsules emerge from the nozzles 12, 14 in a liquid state and are rapidly hardened my means of a powder collection system, a solvent collection bath or similar means. Subsequent to hardening, the microcapsules are dried using any means known in the art, such as solvent evaporation, tumble, or fluid bed drying.

In one embodiment, an acid collection bath is used to rapidly harden the enterically coated microcapsules. The acid collection bath comprises an acidic liquid solvent where the microcapsules are submerged. Due to the non-soluble nature of the enteric coating in an acidic environment, the microcapsules ‘harden’, and then separate from the resulting solvent/water solution. In one embodiment the acid collection bath comprises an acidic liquid solvent, such as lactic acid, glacial acetic acid, citric acid, hydrochloric acid or sulphuric acid; water; a trace amount of polysorbate 80; and, optionally, glycerol. In one embodiment, the acid collection bath comprises glacial acetic acid diluted to 20% with water and a trace amount of polysorbate 80 (Tween® 80). In another embodiment, the acid collection bath comprises about 10% glacial acetic acid, about 10% glycerol, about 80% water and a trace amount of polysorbate 80. Other liquid reaction baths that can be used, depending upon the incorporated gelling agent, include calcium salt solution. The temperature of the liquid bath may be lowered to accelerate capsule hardening to a temperature less than 25° C. Also, the liquid bath may be agitated to prevent capsule agglomeration or sticking using suitable stirring mechanisms well known in the art. The pH of the acid collection bath may range from about 1 to about 4, in one embodiment from about 2 to about 3. The hardened microcapsules are subsequently easily drained of the solvent and dried.

In an alternate embodiment, a powder collection system is used to remove water and harden the shell to produce microcapsules. In particular, a powder collection method utilizing hydrophobic, modified food starch (such as DRY-FLO® supplied by National Starch Company) may be used to harden the microcapsules. Suitable powders for use in the collection system of the present invention have the ability to retain water. The microcapsules are contacted with the powder by any means known in the art, such as pouring the microcapsules on a flat surface pre-coated with powder. The powder coats the capsule surface and water is removed by absorption into the powder. In addition, the powder prevents the capsules from sticking to each other during the collection and drying process. The starch forms a thin coating on the capsule surface and is separated from the capsules by screening, and the moisture in the shell is removed.

Drying of the microcapsules follows, in one embodiment, by solvent evaporation (not shown). The solvent evaporation process includes large dryers that can provide adequate airflow and heat to dry the capsule wall. The water content in the capsule shell may range from about 1% to about 10%, and preferably about 2% to about 5%, by weight of the shell material. Alternatively, the hardened capsules are separated from the solvent media and dried to remove the excess solvent using a tumble drier or fluid bed process. In batch drying, an inert material that is not dense enough to add significant weight to the microcapsule should be used to serve as a spacer while microcapsules dry. The spacer serves to minimize contact between the microcapsules and to prevent agglomeration.

The size range of the microcapsules produced by the above processes may vary from about 200 μm to about 2000 μm. The preferred microcapsule size range is about 500 μm to about 1000 μm. The microcapsule payload may vary from about 10% to about 70% by weight of the microcapsule, and in one embodiment is from about 40% to about 60%, by weight of the microcapsule. The loading is controlled by adjusting the feed rates of the liquid shell and core to provide the desired (after removal of shell solvent) payload.

In another embodiment, the microcapsules can be prepared by a double nozzle, vibratory excitation process which produces a microcapsule comprising two concentric droplets. As with the centrifugal extrusion process, there is great flexibility with regard to selecting suitable shell and core material. The process can be used with nearly all materials that are capable of being liquefied, such as by dissolving of the material in a solvent or melting of a material. Starting materials for use in the double nozzle, vibratory excitation process should have a viscosity of less than 10000 mPa/s, in one embodiment less than 1000 mPa/s. Generally, double nozzle, vibratory excitation allows the material for the inner core can be any material that does not negatively interact with the shell material. Large differences in the densities of the core and shell compositions should be avoided.

To form a microcapsule by the vibratory excitation method, the core and shell materials are pumped from a feed tank, via separate feed lines, to a double concentric nozzle arrangement. Upon exiting the double concentric nozzle arrangement, a composite liquid stream comprising two immiscible concentric components is formed. The liquid stream or streams may be subjected to vibratory action either before entering the double nozzle arrangement, such as while still in the feed lines, while passing through the double nozzle arrangement or after leaving the double nozzle arrangement. Generally a vibration generator is connected either directly or indirectly to the nozzle to generate a vibration to the liquid stream. Potential vibration generators include, but are not limited to, magnetic-induction vibrators, mechanical vibrators, pneumatic vibrators, piezoelectric converters, and electroacoustic converters,

The vibratory action causes oscillation to the stream sufficient to cause the liquid stream to disintegrate or break up into individual, uniform droplets. Surface tension acting on the droplets forms uniform spheres which begin to coagulate during free fall from the nozzle. During free fall the drops coagulate to the point that they are able to maintain integrity upon entering a suitable collection unit. In one embodiment, the collection unit comprises a solidification solution, such as a gaseous and/or liquid medium, which promotes completion of the solidification process. In one embodiment the collection unit comprises an acid collection bath as described above. Distance from the double nozzle arrangement to the surface of the solidification solution may vary depending on the extent of solidification desired. In one embodiment, the distance from the double nozzle arrangement to the surface of the solidification solution may be more than about 10 centimeters. Solidification or coagulation can be further induced by one or more of the following methods, which may be carried during the collection step: cooling, drying or any chemical reaction means.

Preferred embodiments of the present invention are exemplified below. The following examples, however, are in no way intended to limit the scope of the present invention.

### EXAMPLES

### Example 1

Microcapsules containing a lipidic core comprising a mixed glyceride and a surfactant and an enteric shell comprising HPMCP-55 were prepared in accordance with the following composition and centrifugal coextrusion processing parameters.

### Core Composition

| | Amount |
|---|---|
| Components | (% w/w) |
| Partially hydrogenated cotton seed oil (Paramount ® C) | 75 |
| Polyglycolized Glycerides (Gelucire ® 44/14) | 25 |

### Shell Composition

| Components | Amount (% w/w) |
|---|---|
| Water* | 73.0 |
| Sodium Hydroxide | 3.2 |
| HPMCP-55 | 22.4 |
| Glycerine | 1.4 |

Note:
pH adjusted to 5.63 with 10% glacial acetic acid
*Water removed upon drying

### Process Parameters

### Nozzle Specification

Shell Orifice (outer)—1 mm
Core Orifice (inner)—0.5 mm
   Feed Rate (g/min)
Shell (outer orifice)—43 g/min
Core (inner orifice)—22 g/min

### Rotational Speed (RPM)

Centrifugal Head Speed (RPM)—900 RPM

### Collection Media

DRY-FLO® modified starch or Glacial Acetic Acid diluted to 20% w/w with water and trace amount of Tween® 80.

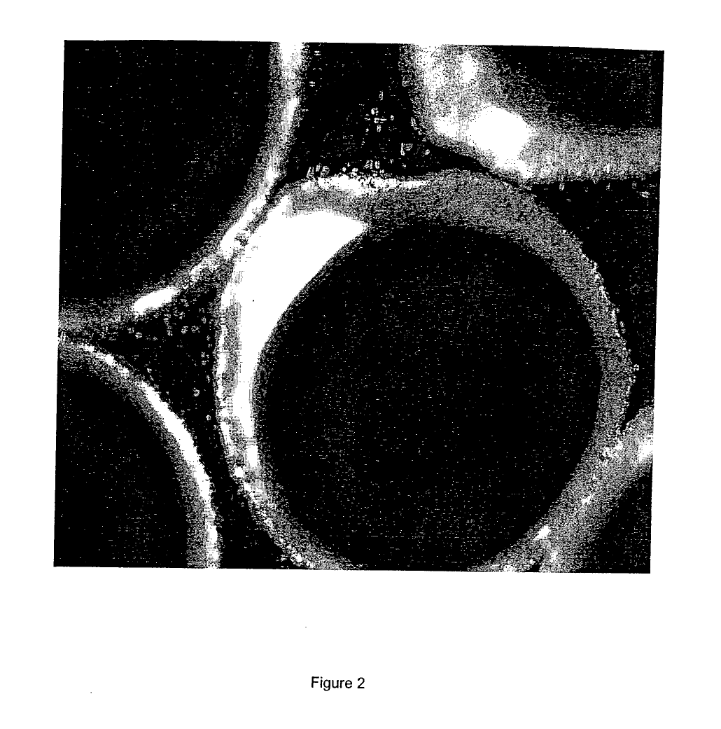

The optical micrographs of the microcapsules of Example 1 are shown in FIG. 2. The microcapsules were spherical, and the particle size of microcapsules ranged from about 500 μm to about 800 μm. The payload of the microcapsules was about 60% of the capsule weight.

### Example 2

Microcapsules containing a lipidic core comprising a medium chain triglyceride and a sparingly water-soluble drug and an enteric shell comprising HPMCP-55 were prepared in accordance with the following composition and centrifugal coextrusion processing parameters. The resulting microcapsule had poor aqueous solubility (<5 μg/mL).

### Core Composition

| Components | Composition (% w/w) |
|---|---|
| Medium Chain Triglyceride (Labrafac ® CC) | 85 |
| Polyglycolized Glycerides (Gelucire ® 44/14) | 10 |
| Drug (SB462795) | 5 |

### Shell Composition

| Components | Composition (% w/w) |
|---|---|
| Water* | 73.0 |
| Sodium Hydroxide | 3.2 |
| HPMCP-55 | 22.4 |
| Glycerine | 1.4 |

Note:
pH adjusted to 5.63 with glacial acetic acid
*Water removed upon drying

### Process Parameters

### Nozzle Specification

Shell Orifice (outer)—1 mm
Core Orifice (inner)—0.5 mm
   Feed Rate (g/min)
Shell (outer orifice)—43 g/min
Core (inner orifice)—22 g/min

### Rotational Speed (RPM)

Centrifugal Head Speed (RPM)—900 RPM

### Collection Media:

DRY-FLO® modified starch or
Glacial Acetic Acid diluted to 20% w/w with water and trace amount of Tween® 80.

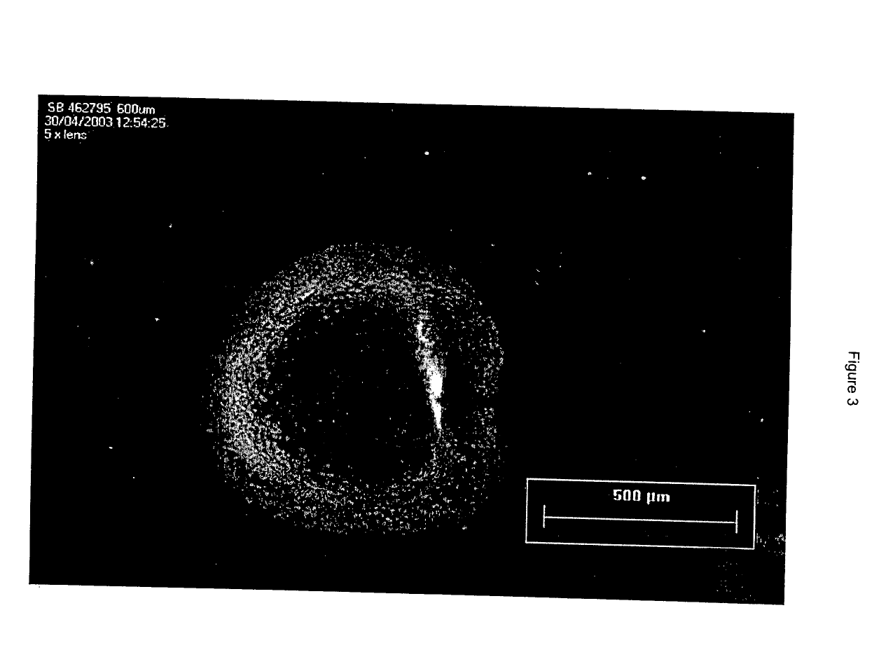

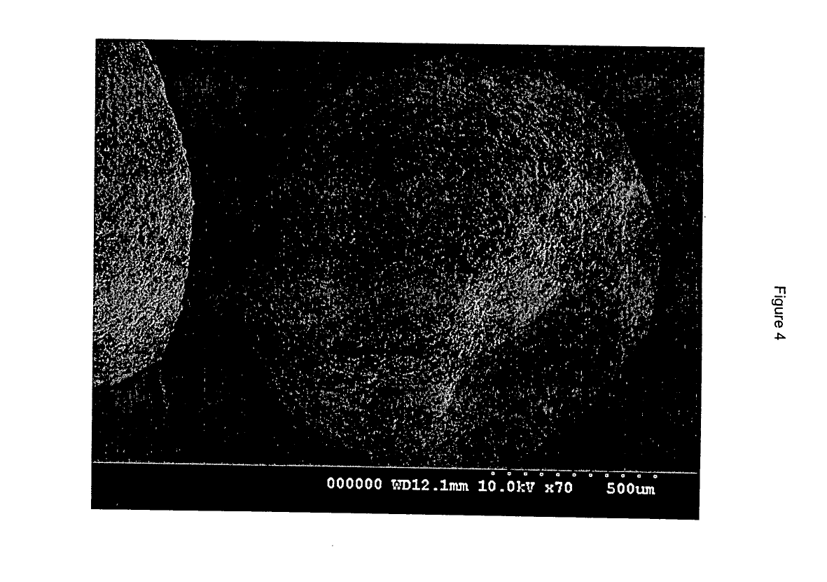

The optical and SEM micrographs of the microcapsules described in Example 2 are shown in FIGS. 3 and 4. The microcapsules were spherical with majority of the microcapsules about 600 μm to about 800 μm in size. Dissolution studies were done on the microcapsules in physiologically relevant media, simulated gastric fluid (0.1 N HCL, pH 1.2, no enzymes added) and simulated intestinal fluid (fed state, pH 5.0), in terms of pH conditions and composition encountered in gastrointestinal tract to better predict release and dissolution characteristics in-vivo.

Dissolution studies were done using a USP III flow-through dissolution apparatus (SOTAX CE 70). In these studies, a predetermined amount of microcapsules (400 mg) was placed in a flow through cell (22.6 mm cell). The flow rate of dissolution medium (@ 37° C.) through the cell was maintained at 8 mL/min. The microcapsules were first exposed to simulated gastric fluid (SGF) for 30 minutes followed by simulated intestinal fluid (SIF) for 1 hour. Samples were collected at predetermined time intervals and analyzed using an HPLC method to determine the release and dissolution characteristics of the microcapsules upon exposure to the two dissolution media at physiological conditions.

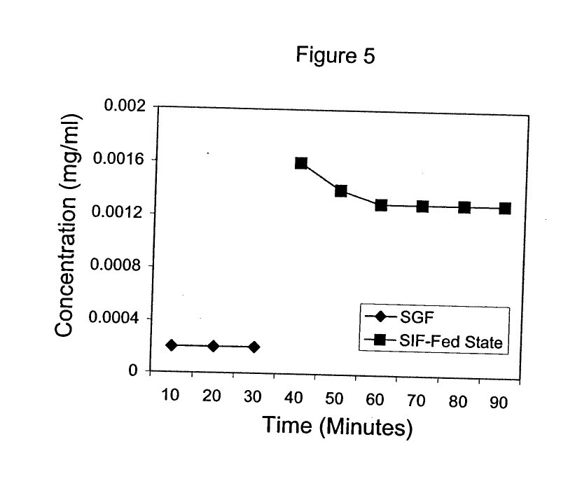

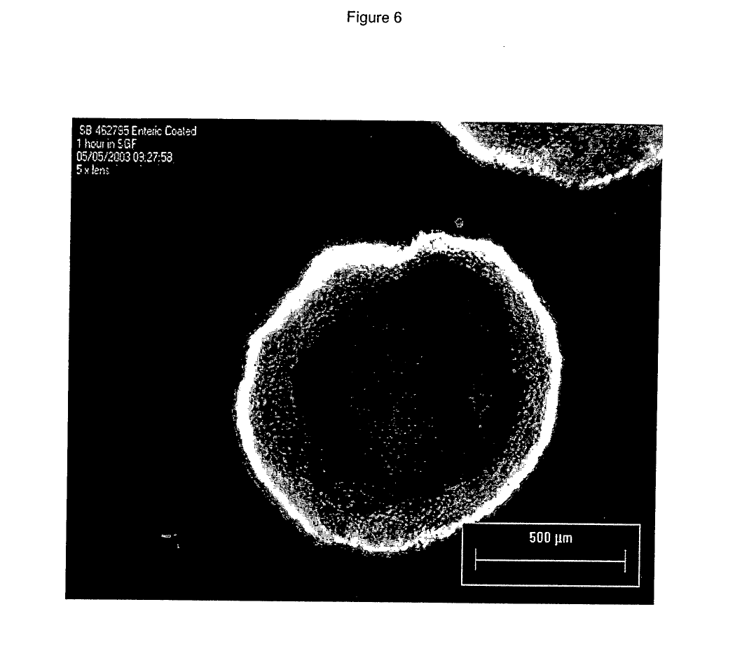

The microcapsules showed negligible release in the dissolution medium at acidic pH (SGF) as summarized in the graph depicted in FIG. 5. The microcapsules showed rapid release and drug solubilization in a dissolution medium that mimics intestinal fluid in terms of pH and composition (SIF) as set forth in FIG. 5. The optical micrographs of the microcapsules upon exposure to SGF showed that the integrity of the capsule was maintained as shown in FIG. 6.

### Example 3

Microcapsules containing a lipidic core and an enteric shell comprising HPMCP-55 are prepared in accordance with the following composition and double nozzle vibratory excitation processing parameters.

### Core Composition

| Components | Composition (% w/w) |
|---|---|
| Miglyol 812 | 90% |
| Active Ingredient | 10% |

### Shell Composition

| Components | Composition (% w/w) |
|---|---|
| Water* | 83.31 |
| HPMCP-55 | 12.08 |
| Glycerine (99.5%) | 1.25 |
| Tween ® 80 | 0.03 |
| NH₃ (25%) | 3.33 |

Note:
Viscosity at 20° C.: 90 mPa/sec

### Process Parameters

### Nozzle Diameter

Shell Orifice (outer)—500 μm
Core Orifice (inner)—300 μm

### Vibration Frequency (Hz)

230 Hz
   Distance from Nozzle to Surface of Solidification Solution (cm)
15 cm

### Solidification Solution

| Components | Composition (% w/w) |
|---|---|
| Acetic Acid or Citric Acid | 9.01 |
| Water | 81.90 |
| Glycerin | 9.01 |
| Tween ® 80 | 0.08 |

Microcapsules are subsequently dried.

### Method of Using

The microcapsules of the present invention can be filled directly into capsule shells or blended with granules containing a different active and then filled into capsule shells suitable for dosing.

The present invention has been described with particular reference to the preferred forms thereof. It will be obvious to one of ordinary skill in the art that changes and modifications may be made therein without departing from the spirit and scope of the present invention and as defined by the following claims.

What is claimed:

1. A method of preparing an active agent for delivery to a selected region in the gastrointestinal tract in a mammalian body comprising the steps of:
encapsulating a lipid-based core having a liquid or solid molecular dispersion
with one or more sparingly water-soluble actives in an enteric polymer shell;
wherein said enteric polymer shell exhibits negligible dissolution in an acidic environment; and
wherein said one or more sparingly water-soluble actives are released from said microcapsule when exposed to an alkaline environment.

2. The method of claim 1, wherein said lipid-based core is encapsulated in said enteric polymer shell by a double nozzle, vibratory excitation process.

3. A method for producing a microcapsule comprising the steps of:
a. providing a first liquid component comprising a lipid-based core material;
b. providing a second liquid component comprising an enteric polymer shell material;
c. forcing said first liquid component through the inner-most nozzle of two concentric vibratory nozzles and concurrently forcing said second liquid component through the outer most of said two concentric vibratory nozzles, thereby producing a composite liquid stream having two concentric liquid components; and
d. causing the composite liquid stream to oscillate by vibratory force sufficient to break said liquid stream into distinct microcapsules having a lipid-based core material encapsulated in said enteric polymer shell material.

4. The method of claim 3 further comprising the step hardening the enteric polymer shell material by immersing said distinct microcapsules into an acid collection bath.

5. The method of claim 4 wherein the acid collection bath has a pH of from about 1 to about 4.

6. The method of claim 5 wherein the acid collection bath has a pH of from about 2 to about 3.

7. The method of claim 6 wherein the acid collection bath is maintained at a temperature of less than about 25° C.

8. A microcapsule prepared by the method of claim 3, wherein said lipid based core material comprises at least one lipidic carrier forming a liquid molecular dispersion and one or more sparingly water-soluble actives and wherein said enteric polymer shell material exhibits negligible dissolution in an acid environment.

9. The microcapsule of claim 8 wherein, said one or more sparingly water-soluble actives is present in said lipid based core material in an amount from about 0.01% to about 20% by total weight of the lipid based core material.

10. The microcapsule of claim 8, wherein said lipid based core material further comprises an ester selected from the group consisting of one or more of medium chain fatty acid esters, long chain fatty acid esters and any combination thereof.

11. The microcapsule of claim 10, wherein said medium chain fatty acid esters and said long chain fatty acid esters are mixed glycerides that have the ability to modulate rigidity of said molecular dispersion.

12. The microcapsule of claim 10, wherein said medium chain fatty acid esters, long chain fatty acid esters and any combinations thereof are present in said lipid based core material in an amount from about 75% to about 99.99% by total weight of the lipid based core material.

13. The microcapsule of claim 8, wherein said lipid based core material further comprises one or more lipid based surfactants.

14. The microcapsule of claim 13, wherein said one or more lipid based surfactants are present in said lipid based core material in an amount from about 0.01% to about 25% by total weight of the lipid based core material.

15. The microcapsule of claim 8, wherein said lipid based core material further comprises one or more solubilization enhancers.

16. The microcapsule of claim 15, wherein said one or more solubilization enhancers are present in said lipid based core material in an amount from about 0.01% to about 10% by total weight of the lipid based core material.

17. The microcapsule of claim 8, wherein said lipid based core material has a payload from about 10% to about 80% based on the total weight of the microcapsule.

18. The microcapsule of claim 8, wherein said enteric polymer shell material is formed from one or more of the polymers selected from the group consisting of cellulose acetate phthalate, hydroxypropyl methylcellulose phthalate, hydroxypropyl methycellulose acetate succinate, alkali-soluble acrylic copolymer, polyvinyl acetate phthalate, alginates, or combinations, thereof.

19. The microcapsule of claim 8, wherein said enteric polymer shell material further comprises a plasticizer.

20. The microcapsule of claim 8, wherein said enteric polymer shell material further comprises a pigment.
